(19) United States
(12) Plant Patent Application Publication
(10) Pub. No.: US 2003/0172423 P1
(43) Pub. Date: Sep. 11, 2003

(54) L96-117 sweetpotato

(76) Inventors: Don R. LaBonte, Baton Rouge, LA (US); Christopher A. Clark, Baton Rouge, LA (US); James M. Cannon, Baton Rouge, LA (US); Paul W. Wilson, Gonzales, LA (US); Arthur A. Villordon, Monroe, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 10/091,255

(22) Filed: Mar. 5, 2002

(51) Int. Cl. A01H 5/00

(52) U.S. Cl. PLT/258

(57) ABSTRACT

A new variety of sweetpotato identified as “L96-117 sweetpotato” is disclosed having superior processing qualities and a high total-sugar content. L96-117 is characterized by an intense orange flesh and an elongated root.

This invention pertains to a variety of sweetpotato having superior processing qualities and a high total-sugar content.

This new variety is identified as “L96-117 Sweetpotato” (“L96-117”). L96-117 is a sweetpotato variety, Ipomoea batatas (L.) Lam., developed from a seedling produced in a polycross nursery. L96-117 demonstrates both superior processing qualities and high total-sugar content as compared to other available sweetpotato varieties. L96-117 is characterized by an intense orange flesh and an elongated root.

Sweetpotatoes, unlike Irish potatoes (Solanum tuberosum), are not tuber propagated plants. A “tuber” is a short, thickened portion of an underground branch. Along a tuber are found “eyes,” each of which comprises a ridge bearing a scale-like leaf (analogous to a branch leaf) having minute meristematic buds in the axial of the leaf. By contrast, sweetpotato roots are developmentally and anatomically true roots, lacking meristematic buds, and are not derived from an underground branch. Sweetpotatoes do not form tubers.

### BRIEF DESCRIPTION OF THE DRAWING

FIG. 1 is a color photograph of the fleshy root form of the novel variety of sweetpotato identified as “L96-117 sweetpotato.”

FIG. 2 is a color photograph of the fleshy root form of the sweetpotato variety identified as “Beauregard sweetpotato.”

FIG. 3 is a color photograph of the canopy biomass of the novel variety of sweetpotato identified as “L96-117 sweetpotato.”

The file of this patent contains at least one photograph executed in color. Copies of this patent with color drawing(s) will be provided by the Patent and Trademark Office upon request and payment of the necessary fee.

L96-117 was developed by the Louisiana Agricultural Experiment Station in Baton Rouge, La., to provide a cultivar with characteristics similar to the Beauregard sweetpotato, but with improved processing qualities. L96-117 originated as a seedling from a polycross nursery of the previous year. The female lineage of L96-117 is L91-189 and Beauregard. (The female parent of Beauregard is L78-21.) The male parent of L96-117 is unknown. All parents were derived from the sweetpotato breeding program at the Louisiana Agricultural Experiment Station.

L96-117 roots were stored during the winter at the Louisiana Agricultural Experiment Station (Sweetpotato Research Station) in Chase, La. During the following spring, L96-117 was planted and produced approximately 8-10 sprouts, which were cut and transplanted successfully for asexual reproduction.

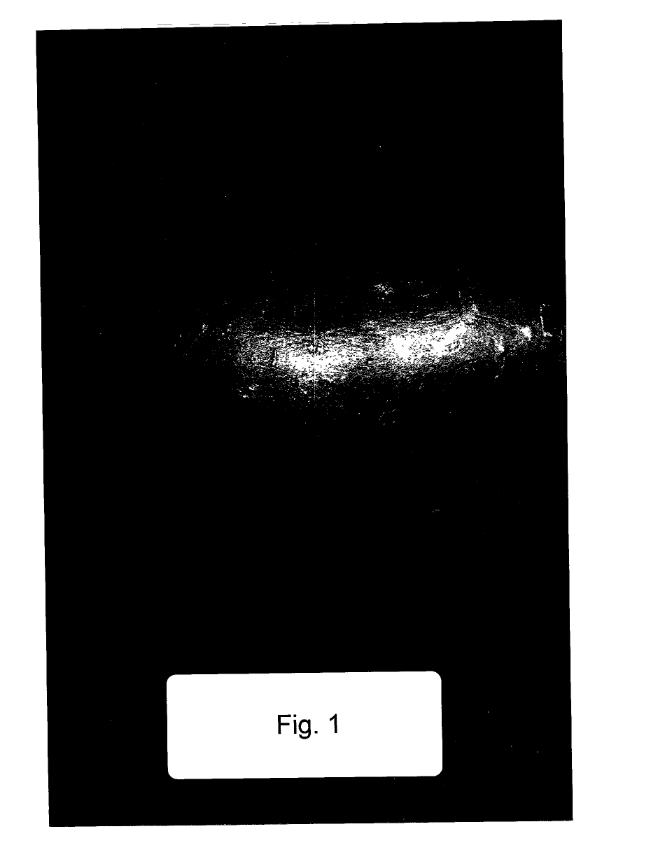

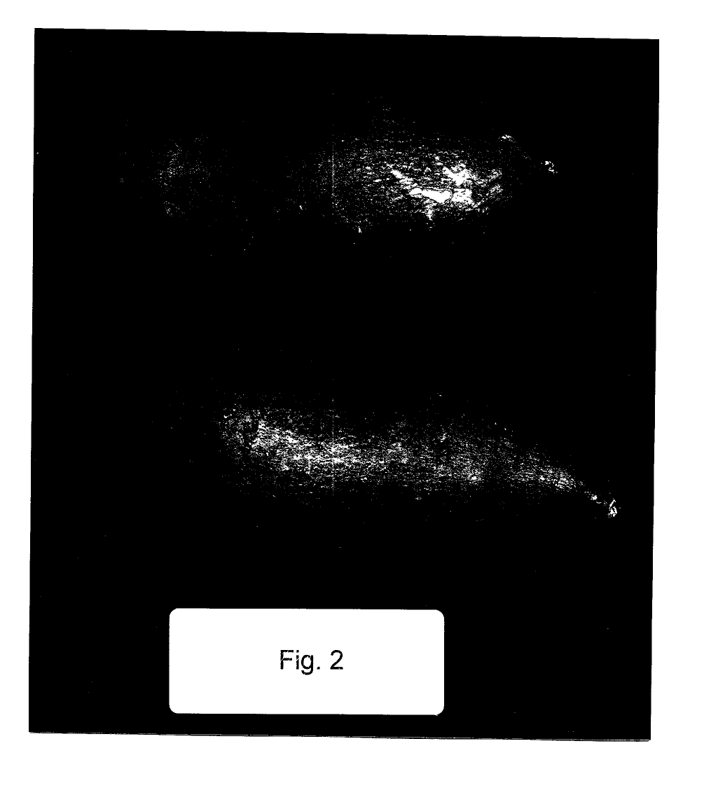

FIG. 1 depicts the fleshy root form of the L96-117 sweetpotato. Skin varies in color from light to dark rose, and is typically darker than Beauregard at harvest. (Skin color lightens in storage.) See Beauregard as depicted in FIG. 2. Skin is smooth, similar to that of Beauregard; however, the flesh is more uniformly orange than Beauregard. The cortex is 3-4 mm in depth.

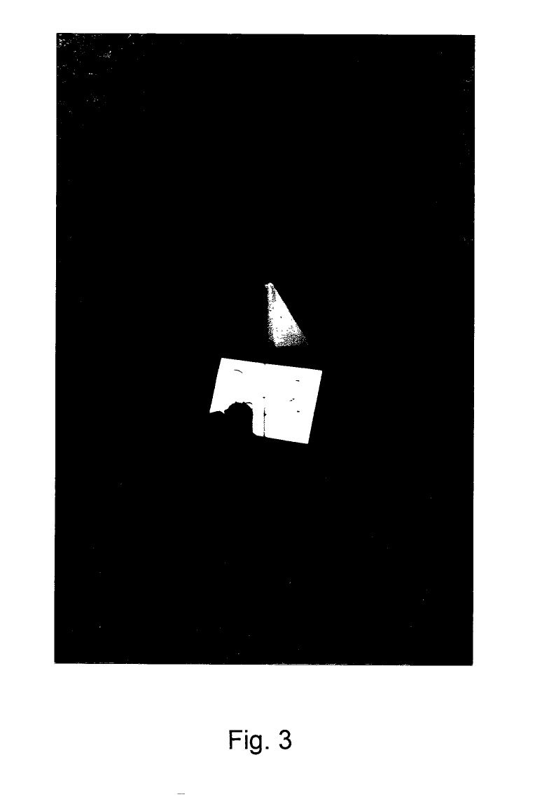

FIG. 3 depicts the canopy biomass of the L96-117 sweetpotato. L96-117 sweetpotato has purple-stemmed vines with green mottling that begin fading to green about 38 cm from the apex. The appearance of the canopy biomass falls between varieties Jewel and Beauregard, and covers the surface of the soil. Unfolded immature leaves are purple-edged with purple abaxial veins. The abaxial veins remain mostly purple as they mature, while the adaxial veins are mostly green with some purple. Mature leaves have an acute apex and a cordate base. The leaves vary between 1-3, slight to deep lobes. The petiole is light purple on young, maturing leaves within 20 cm of the vine apex, and gradually changes to green. A purple joint is located at the intersection of the petiole and the leaf. The node from which the petiole attaches to the stem is also green. Storage roots are elongated without any lobing.

Colorimetric evaluations using the Average Hunter Chromacity values (hue (L), value (a), and chroma (b)) of skin, cortex and flesh for both L96-117 and Beauregard storage roots, at harvest, are shown in Table 1. Munsell® color equivalents for hue (L), value (a), and chroma (b) have also been provided in Table 1, using conversion software provided by Munsell at <http://munsell.com>.

TABLE 1

| | Variable/ | L | a | b |
|---|---|---|---|---|
| | Cultivar | (lightness/darkness) | (red/green) | (blue/yellow) |
| Skin | L96-117 | 57.2 ± 10.1 | 27.7 ± 3.2 | 25.3 ± 3.0 |
| | | (1.41 YR)^{z} | (5.55) | (6.28) |
| | Beauregard | 52.3 ± 2.1 | 19.4 ± 1.1 | 27.8 ± 4.7 |
| | | (3.18 YR) | (5.07) | (6.05) |
| Cortex | L96-117 | 68.2 ± 2.2 | 37.5 ± 2.9 | 52.4 ± 7.1 |
| | | (1.88) | (6.65) | (11.80) |
| | Beauregard | 69.8 ± 1.7 | 34.6 ± 3.2 | 42.5 ± 6.3 |
| | | (1.10 YR) | (6.82) | (10.38) |
| Flesh | L96-117 | 66.1 ± 0.6 | 34.7 ± .68 | 43.6 ± .72 |
| | | (1.29 YR) | (6.44) | (10.43) |
| | Beauregard | 68.4 ± 2.5 | 30.1 ± 2.1 | 39.1 ± 2.8 |
| | | (1.74 YR) | (6.67) | (9.28) |

^{z}Data in parentheses represent Munsell ® color equivalents for hue (L), value (a), and chroma (b).

### EXAMPLE 1

Tests Conducted

To confirm that L96-117 was anew variety, controlled tests (e.g., pathogen responses and yield) were conducted at the Louisiana Agricultural Experiment Station in Baton Rouge, La. As an ancestor of L96-117, Beauregard was selected for comparison tests. Diseases that commonly affect the growth of sweetpotatoes were selected to test for pathogen responses in both varieties. Scions of L96-117 and Beauregard reacted similarly to most diseases evaluated in the controlled tests. L96-117 was less resistant to Fusarium wilt, caused by Fusarium oxysporum Schlect. f. sp. batatas (Wollenw.) Snyd. & Hans., than was Beauregard. However, L96-117 exhibited higher resistance to soil rot, caused by Streptomyces ipomoeae (Person and Martin) Waksman & Henrici., than did Beauregard.

Nematode reproduction was measured in greenhouse tests. L96-117 exhibited higher resistance to the southern root-knot nematode, Meloidogyne incognita (Kofoid and White, 1919) Chitwood, 1949, race-3, than Beauregard. Both L96-117 and Beauregard were susceptible to the reniform nematode, Rotylenchulus reniformis Linford & Oliveira, 1940. L96-117 and Beauregard were both resistant to the development of internal cork, a disease presumably caused by a virus (unknown). L96-117 and Beauregard exhibited similar resistance to Fusarium root rot caused by Fusarium solani (Mart.) Sacc. Emend. Snyd. & Hans. L96-117 exhibited higher resistance to bacterial root rot, caused by Erwinia chrysanthemi Burkholder, McFadden and Dimock, 1953, than did Beauregard. L96-117 exhibited lower resistance to Rhizopus stolonifer (Her. ex. Fr.) Lind., than did Beauregard. Circular spot, caused by Sclerotium rolfsii Sacc., varied from a low to a high incidence in both L96-117 and Beauregard.

No formal trials have been conducted to date on L96-117 for insect pests. L96-117 does not appear to show any novel insect resistance. Both L96-117 and Beauregard show similar levels of susceptibility to important insects pests, most notably the banded cucumber beetle, Diabrotica balteata LeConte, and white grub, Phyllophaga ephilida Say.

To determine yield production, complete-block trials using four replications of L96-117 and Beauregard each were conducted at two different Louisiana Agricultural Experiment Station locations, Burden Research Plantation in Baton Rouge, La. and the Sweetpotato Research Station in Chase, La. Both L96-117 and Beauregard were transplanted in randomized complete-block trials at 31, 36, and 41 cm spacings, in Loring silt loam soil at the Burden Research Plantation and Gilbert silt loam soil at the Sweetpotato Research Station. Each block/plot was fertilized with 250 pounds per acre of nitrogen, P₂O₅, and K₂O (about 250 pounds per acre of 13% N, 13% P₂O₅, and 13% K₂O, 13-13-13 mixed fertilizer). L96-117 was compared to Beauregard at early and middle transplanting dates at each location beginning in June. Average yields were measured for the following grades of roots: U.S. #1 (51-89 mm in diameter, 76-229 mm long); Canner (25-51 mm in diameter, 51-178 mm long); and Jumbo (larger than U.S. #1 in diameter, length or both, and without objectionable defects).

Early transplanting date trials were conducted at the Burden Research Plantation. L96-117 and Beauregard were transplanted on June 13 and harvested on October 11 (120 days after planting). Average yields, measured as Mg·ha⁻¹, are shown in Table 2.

TABLE 2

| Selection | | | | |
|---|---|---|---|---|
| (spacing, in cm) | US#1 | Canners | Jumbos | TMY^{‡} |
| L96-117 (41) | 22.8a^{†} | 5.5b | 1.2bc | 29.5ab |
| L96-117 (36) | 22.8a | 5.7ab | 2.8abc | 31.3a |
| L96-117 (31) | 21.9a | 5.8ab | 2.6bc | 30.2ab |
| Beauregard (41) | 22.4a | 5.3b | 4.0ab | 31.7a |
| Beauregard (36) | 22.8a | 6.4ab | 5.6a | 34.8a |
| Beauregard (31) | 25.1a | 6.8ab | 4.2ab | 36.0a |
| Least Significant | 5.6 | 2.1 | 3.0 | 6.3 |
| Difference | | | | |
| LSD (P<0.05) | | | | |

^{†}Average yields of varieties followed by a common letter do not differ significantly (P<0.05) according to Duncan's Multiple Range Test.
TMY^{‡} = total marketable yield

Middle transplanting date trials were also conducted at the Burden Research Plantation. L96-117 and Beauregard were transplanted on June 22 and harvested on October 27 (127 days after planting). Average yields (Mg·ha⁻¹) of L96-117 and Beauregard are shown in Table 3.

TABLE 3

| Selection | | | | |
|---|---|---|---|---|
| (Spacing, in cm) | US#1 | Canners | Jumbos | TMY^{‡} |
| L96-117 (41) | 13.4a^{†} | 3.4b | 1.7a | 18.5ab |
| L96-117 (36) | 11.4a | 5.8ab | 3.2a | 20.3ab |
| L96-117 (31) | 10.5a | 7.3ab | 2.7a | 20.6ab |
| Beauregard (41) | 7.6a | 4.7ab | 4.6a | 16.9b |
| Beauregard (36) | 14.2a | 3.3b | 4.1a | 21.6ab |
| Beauregard (31) | 17.1a | 13.4a | 7.9a | 38.5a |
| Least Significant | 7.5 | 7.0 | 6.1 | 15.5 |
| Difference | | | | |
| LSD (P<0.05) | | | | |

^{†}yields of varieties followed by a common letter do not differ significantly (P<0.05) according to Duncan's Multiple Range Test.
TMY^{‡} = total marketable yield

Early transplanting date trials were also conducted at the Sweetpotato Research Station. L96-117 was transplanted on June 1 and harvested on September 27 (118 days after planting). (Beauregard was not included in this transplanting trial.) Average yields (Mg·ha⁻¹) by grade are shown in Table 4.

TABLE 4

| Selection | | | | |
|---|---|---|---|---|
| (Spacing, in cm) | US#1 | Canners | Jumbos | TMY^{‡} |
| L96-117 (21) | 30.3 | 11.5 | 4.4 | 41.9 |
| L96-117 (36) | 25.6 | 11.4 | 1.0 | 37.1 |
| L96-117 (41) | 25.2 | 10.7 | 0.8 | 36.0 |
| Least Significant | 7.8 | 4.1 | 5.4 | 7.5 |
| Difference | | | | |
| LSD (P<0.05) | | | | |

TMY^{‡} = total marketable yield

Middle transplanting date trials were also conducted at the Sweetpotato Research Station. L96-117 and Beauregard were transplanted on July 12, and harvested on October 31 (111 days after planting) using 31,36 and 41 cm spacings. Average yields (Mg·ha⁻¹) by grade are shown in Table 5.

TABLE 5

| Selection | | | | |
|---|---|---|---|---|
| (Spacing, in cm) | US#1 | Canners | Jumbos | TMY^{‡} |
| L96-117 (41) | 4.5c | 9.1b | 1.0a | 13.6c |
| L96-117 (36) | 5.4bc | 11.7ab | 0.9a | 17.1bc |
| L96-117 (31) | 4.6c | 11.7ab | 2.4a | 16.2c |
| Beauregard (41) | 10.1a | 12.1ab | 0.6a | 22.1a |
| Beauregard (36) | 9.2ab | 11.5ab | 1.6a | 20.9ab |
| Beauregard (31) | 9.2ab^{†} | 13.8a | 0.6a | 23.1a |
| Least Significant | 4.2 | 4.8 | 3.2 | 4.3 |
| Difference | | | | |
| LSD (P<0.05) | | | | |

^{†}Average yields of varieties followed by a common letter do not differ significantly (P<0.05) according to Duncan's Multiple Range Test.
TMY^{‡} = total marketable yield

As shown in Tables 2-5, L96-117 produced yields comparable to Beauregard at early transplanting dates (95% of Beauregard for U.S. #1 grade; 91% of Beauregard for total marketable yield). Spacing had no significant effect on yield. (At later planting dates, L96-117 had yields slightly less than those of Beauregard, but still competitive. Replicated plots on sweetpotato production farms have not shown any predisposition of L96-119 to low yield characteristics in late plantings.)

L96-117 was also compared to Beauregard for physiological attributes. Using replicates of seven month stored sweetpotato roots, it was determined that L96-117 has an Alcohol Insoluble Solid (AIS) content (i.e., starch) of 14.5% (fresh wt. basis). By comparison, Beauregard has an AIS content of 11.4%. (AIS content of freshly harvested roots for L96-117 (25.2%) was similar to that of Beauregard (23.2%)). L96-117 had higher total sugars (6.7%, 10 g fresh wt. basis) as compared to that of Beauregard (5.2%) for seven month stored sweetpotato roots. Puree-processed, freshly harvested roots of L96-117 had higher total sugar content (84 mg/gm fresh wt. basis) than did a comparable sample of Beauregard (37 mg/gm fresh wt. basis).

L96-117 produces plants (sprouts) at an earliness and quantity similar to Beauregard. Days to harvest are similar to, and sometimes greater than, Beauregard. The roots of L96-117 are more elongated than those of Beauregard. Yield of total and number one grade roots is slightly less than that of Beauregard. L96-117's primary expected use is as a processor variety. Root length of L96-117 makes it less desirable for the fresh sweetpotato market. However, L96-117's high sugar content and intense orange flesh make it well suited for production of a superior puree for uses such as baby food puree.

We claim:

1. A sweetpotato plant substantially as described and illustrated in the specification herein.
